(19) United States
(12) Patent Application Publication
(10) Pub. No.: US 2011/0098360 A1
(43) Pub. Date: Apr. 28, 2011

(54) ADAMANTANE DERIVATIVE FOR INHIBITING TOXICITY OF AMYLOID OLIGOMER

(75) Inventors: Min Yung Lee, Seoul (KR); So-Yeop Han, Seoul (KR); Jun Mo Chung, Seoul (KR); Pyung Lim Han, Seoul (KR); Jung Min Koh, Seoul (KR); Ha Yan Oak, Seoul (KR); Eun Kyung Hwang, Yongin-si (KR); In Sun Baek, Seoul (KR)

(73) Assignee: NANODIAMOND, INC., Seoul (KR)

(21) Appl. No.: 12/935,244

(22) PCT Filed: Mar. 9, 2009
(86) PCT No.: PCT/KR2009/001147

§ 371 Date: Dec. 13, 2010

(30) Foreign Application Priority Data
Mar. 28, 2008 (KR) 10-2008-0029243

(51) Int. Cl. A61K 31/132; C07C 211/19; A61P 25/28; A61P 25/16; A61P 25/00; A61P 27/02

(52) U.S. Cl. 514/659; 564/456

(57) ABSTRACT

Disclosed is a pharmaceutical composition containing a compound useful for inhibiting neurotoxicity caused by beta amyloid. The pharmaceutical composition of the present disclosure contains 1,3,5,7-tetrakis(aminomethyl)adamantane, an analogous compound thereof or a salt thereof as an active ingredient. The inventors have studied methods for reducing the toxicity of beta amyloid oligomers based on the formation mechanism of dodecamers in consideration of the fact that especially the dodecamers from among the beta amyloid oligomers exhibit a significant activity as a toxin for synapses and neurons in cranial nerve diseases. The inventors have confirmed that the disclosed compound can induce structural epitope deformation of the dodecamer and thereby reduce toxicity of the beta amyloid oligomers. The pharmaceutical composition containing the compound is useful for preventing and treating cranial nerve diseases developed by the toxicity of beta amyloid oligomers, for example, Alzheimer's disease, Parkinson's disease, Huntington's disease, macular degeneration, prion disease, and the like (see FIG. 1).

### TECHNICAL FIELD

The present disclosure relates to a use of an amine derivative of adamantane, which has a useful medicinal effect, or a salt thereof, and a pharmaceutical composition containing the compound. Being capable of reducing the toxicity of beta amyloid oligomers by inducing structural deformation of the beta amyloid oligomers which play important roles in nerve diseases including Alzheimer's disease, Parkinson's disease, Huntington's disease, macular degeneration, prion disease, and the like, the adamantane derivative or the salt thereof according to the present disclosure may be useful as an agent for preventing or treating the diseases.

### BACKGROUND ART

The human brain contains about 14 billion neurons and it is known that approximately 50,000 are lost every day. The reduction of the brain cells is much faster in patients with Alzheimer's disease (AD). In the brain of the AD patients, insoluble beta amyloid (amyloid-β; Aβ) fibrils are observed as spherical plaques. Thus, the amyloid hypothesis was proposed on the presumption that the beta amyloid fibrils would be related with neuronal death in AD patients (Hardy et al., Science 256, 184-185, 1992). However, as experimental data reveal that the beta amyloid fibrils themselves are not closely related to cytotoxicity, the research on Alzheimer's disease has reached a tuning point. Later, the amyloid oligomer hypothesis was newly proposed as it was known that soluble beta amyloid oligomers, not the less soluble fibrils, have cytotoxicity (Hardy et al., Science 297, 353-356, 2002).

In the early studies about the mechanism of Alzheimer's disease, it was presumed that Aβ monomers are aggregated to form oligomers, which then grow into larger aggregates such as protofilaments or fibrils. However, according to more recent researches, it seems that soluble Aβ oligomers and fibrils are aggregated via an independent path. This means that AR oligomers are not an indispensable intermediate in the formation of fibrils. Strong evidences are reported showing that Aβ oligomers exhibit much stronger neuronal toxicity than fibrils in Alzheimer's disease. Based on these findings, the focus of the study on cranial nerve diseases is shifting from the Aβ fibrils to the Aβ oligomers (Haass et al., Nature Reviews Molecular Cell Biology 8, 101, 2007; Barghorn et al., J. Neurochemistry 95, 834, 2005).

Among Aβ, Aβ₁₋₄₂ consisting of 42 amino acids account for most of the plaques detected in patients with cranial nerve diseases although it is excreted much less (about 10%) than Aβ₁₋₄₀, which consists of 40 amino acids. This is because Aβ₁₋₄₂ has a strong propensity to aggregate and form oligomers. The toxic Aβ₁₋₄₂ oligomers exist in vivo in various forms. The various forms of the oligomers are referred to as amyloid β-derived diffusible ligand (ADDL) (Lambert et al., Proc. Natl. Acad. Sci. USA 95, 6448-6453, 2001), spherical oligomer (Kayed et al., Science 300, 486-489, 2003), globulomer (Barghorn et al., J. Neurochem. 95, 834-847, 2005), Aβ*56 (Lesne et al., Nature 440, 352-357, 2006), and so forth.

The most prevalent among the Aβ₁₋₄₂ oligomers is the 12-mer (dodecamer; hereinafter, (Aβ)₁₂). Among them, (Aβ)₁₂ is drawing a lot of attentions because it has the most general structural epitope with a very strong toxicity for synapses and neurons. However, it is still not clear how (Aβ)₁₂ induces damage of synapses and neurons and, thus, there is no effective therapy available for the development of cranial nerve diseases in the early stage. Presumably, treatment options for diseases related with (Aβ)₁₂ may lie in suppression of the formation of the oligomers, destruction of already formed oligomers, or reduction of toxicity of the oligomers.

In cellular level, there are disagreements as to where (Aβ)₁₂ is formed. Through an in vivo experiment, Lesne et al. clearly observed that the (Aβ)₁₂ oligomers are formed outside the cells (Lesne et al., Nature 440, 352, 2006). In contrast, only the trimer (Aβ)₃ was found in the cells. Thus, it was presumed that (Aβ)₃ produced inside the cells are excreted to outside and then forms larger oligomers such as (Aβ)₆, (Aβ)₉ and (Aβ)₁₂. They reported that, among these oligomers, (Aβ)₁₂ is the major cause of malfunctioning of synapses and neurons. On the contrary to this, Klein et al. reported that they observed (Aβ)₁₂ inside neurons (Klein et al., Trends. Neurosci. 24, 219, 2001). These seemingly contradictory observations imply that (Aβ)₁₂ formed outside the cells may be transferred into the neurons by a plasma membrane receptor, thereby leading to a dynamic equilibrium inside and outside the cell.

(Aβ)₁₂ is a nano-sized toxin extremely harmful to the neuronal functions. Although much remains to be elucidated about the neurotoxicity of (Aβ)₁₂, a lot of mechanisms have been proposed. Examples include formation of ion channels on the cell membrane, malfunctioning of mitochondria, and production of reactive oxygen species. The neurotoxicity of the nano-sized amyloid oligomer seems to be caused by its specific structure allowing it to act as a ligand for the synapse.

In aspects of prevention and treatment, it is much easier to deal with toxic substances outside the cells than those inside the cells. It is because there is no concern of endocytosis of the candidate substance. However, with regard to an agent for preventing or treating or a method for treating the nerve diseases, a compound capable of effectively reducing the toxicity of amyloid oligomers for synapses and neurons, especially in regard to dodecamer formation of the amyloid oligomer, has not been reported as yet.

### DETAILED DESCRIPTION OF THE DISCLOSURE

### Technical Problem

The inventors aim at developing a new therapeutic agent for cranial nerve diseases such as Alzheimer's disease based on the beta amyloid oligomer hypothesis. Currently available Alzheimer's disease drugs include donepezil (Aricept), galantamine (Reminyl), Exelon, tacrine, etc. as acetylcholinesterase inhibitors and memantine, etc. as a N-methyl-D-aspartic acid (NMDA) receptor antagonist. Although they slow the progression of Alzheimer's disease or temporarily improve cognitive function, they do not cure the disease. The present disclosure is directed to providing a use of a specific compound or a salt thereof effective in reducing toxicity of various beta amyloid oligomers including the dodecamer (Aβ)₁₂ for synapses and neurons based on the formation mechanism of (Aβ)₁₂, in consideration of the fact that especially (Aβ)₁₂ from among the beta amyloid oligomers exhibits a significant activity as a toxin for synapses and neurons in cranial nerve diseases such as Alzheimer's disease, and a pharmaceutical composition containing the same.

### Technical Solution

In order to develop a compound capable of reducing the toxicity of beta amyloid oligomers by inducing structural deformation of the beta amyloid oligomer upon contact therewith, the inventors selected adamantane as starting material. Adamantane is a highly symmetrical molecule with a backbone having T_{d} symmetry according to group theory. The bridge carbons at 1-, 3-, 5- and 7-positions are reactive and may be substituted.

The present disclosure is based on a compound having the adamantane backbone as a dendritic core and having 4n (n=1, 2, . . . ) amines as terminal functional groups. Such an adamantane-based dendrimer (AD) compound is named as AD-(NH₂)₄ₙ (n=1, 2 or 3). The AD compound may have one or more branching unit (s) as defined generally in dendrimers.

(Aβ)₁₂ is a large aggregate with a molecular weight of 56 kDa and a diameter of 6-7 nm. In the present disclosure, the dendrimer is employed as a motif because it seems difficult to reduce the toxicity of beta amyloid oligomer through molecular interactions only. The dendrimer consists of a central core, a branching unit and surface functional groups. Since a dendrimer molecule has a tree-like structure, even a relatively simple molecule may have many functional groups. Because (Aβ)₁₂ is formed from four trimers (Aβ)₃, it is expected to interact strongly with the highly symmetric adamantane-based dendrimers.

In an aspect, the present disclosure provides a use of a 1,3,5,7-tetrakis(aminomethyl)adamantane compound shown in FIG. 1, as a typical example of AD-(NH₂)₄ₙ, or a pharmaceutically acceptable salt thereof.

The 1,3,5,7-tetrakis(aminomethyl)adamantane, an analogous compound thereof defined by AD-(NH₂)₄ₙ (n=1, 2 or 3) or a pharmaceutically acceptable salt thereof may reduce toxicity of a beta amyloid oligomer that induces Alzheimer's disease, Parkinson's disease, Huntington's disease, macular degeneration and prion disease.

In another aspect, the present disclosure provides a pharmaceutical composition containing 1,3,5,7-tetrakis(aminomethyl)adamantane, an analogous compound thereof defined by AD-(NH₂)₄ₙ (n=1, 2 or 3) or a pharmaceutically acceptable salt thereof as an agent for reducing toxicity of the beta amyloid oligomer.

In an embodiment, the pharmaceutical composition may prevent or treat a disease selected from a group consisting of Alzheimer's disease, Parkinson's disease, Huntington's disease, macular degeneration and prion disease.

In another embodiment, the pharmaceutical composition may contain a hydrochloride of 1,3,5,7-tetrakis(aminomethyl)adamantane or the analogous compound thereof defined by AD-(NH₂)₄ₙ (n=1, 2 or 3).

### ADVANTAGEOUS EFFECTS

In accordance with the present disclosure, there are provided a use of 1,3,5,7-tetrakis(aminomethyl)adamantane, an analogous compound defined by AD-(NH₂)₄ₙ (n=1, 2 or 3) or a salt thereof as an agent for reducing toxicity of beta amyloid oligomers, and a pharmaceutical composition containing the same. The compounds or the salts thereof have a highly symmetrical structure. While having a strongly hydrophobic core, they are soluble in water because of external amine moieties. The compounds reduce toxicity of beta amyloid oligomers by inducing structural deformation of (Aβ)₁₂ having a strong toxicity for synapses and neurons and, thus, can be used as an agent for preventing and treating cranial nerve diseases including Alzheimer's disease.

### BRIEF DESCRIPTION OF THE DRAWINGS

FIG. 1 shows a molecular structure of 1,3,5,7-tetrakis(aminomethyl)adamantane.

FIG. 2 shows molecular structures of three adamantane derivatives according to embodiments of the present disclosure.

FIG. 3 shows a CD spectrum of (Aβ)₁₂ dissolved in HEPES buffer.

FIG. 4 shows schematic representations of an Aβ monomer, an Aβ trimer and an Aβ dodecamer obtained from conformational and MD computation of an Aβ₁₋₄₂ monomer.

FIG. 5 shows CD spectra of (Aβ)₁₂ in the presence of adamantane derivatives.

FIG. 6 shows real-time FLIM images of a single hippocampal cell treated with (Aβ)₁₂.

FIG. 7 shows fluorescence images of a live H19-7 cell in the presence of (Aβ)₁₂ and adamantane derivatives.

FIG. 8 shows an aspect ratio of a single live H19-7 cell in the presence of (Aβ)₁₂ and adamantane derivatives in an FLIM experiment.

FIG. 9 shows a release curve depicting LTP induced by high-frequency stimulation to a mouse hippocampal slice across the CA1 region as percentage of a baseline potential.

FIG. 10 shows a sweep curve of a mouse hippocampal slice across the CA1 region before (30 min) and after (90 min) applying high-frequency stimulation to the hippocampal slice.

FIG. 11 shows an fEPSP slope 1 hour after applying high-frequency stimulation to a hippocampal slice.

FIG. 12 shows a result of in vivo experiment on Tg-APPswe/PS1dE mice.

### BEST MODE

The inventors have searched for relatively small molecules capable of reducing toxicity of (Aβ)₁₂. For this, they have searched molecules that can deform the nano-sized epitope of (Aβ)₁₂.

As a result, they have found out that an adamantane derivative 1,3,5,7-tetrakis(aminomethyl)adamantane or a salt thereof has such a function, and have confirmed that the compound and the salt thereof may be used to treat cranial nerve diseases such as Alzheimer's disease. Specific examples of the salt may include acid addition salts with inorganic acid salts such as hydrochloric acid, hydrobromic acid, hydroiodic acid, sulfuric acid, nitric acid, phosphoric acid, etc., organic acids such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, etc., or acidic amino acids such as aspartic acid, glutamic acid, etc.

The compound and the salt thereof of the present disclosure are not limited to those described in Examples, and include 1,3,5,7-tetrakis(aminomethyl)adamantane, an analogous compound thereof defined by AD-(NH₂)₄ₙ (n=1, 2 or 3) and a pharmaceutically acceptable salt thereof.

The compound or the salt thereof has a highly symmetrical structure. While having a strongly hydrophobic core, it has hydrophilicity because of the 4n external amine moieties and, thus, is soluble in water in various pH's.

The compound of the present disclosure may be prepared using various known synthesis methods in consideration of the properties of its backbone and substituents. Some functional groups may be protected using appropriate protecting groups in starting material or intermediate stages for effective preparation. Later, the protecting groups may be removed to obtain the desired compound. Hereinafter, specific examples of the compound of the present disclosure will be described.

### EXAMPLES

### Preparation of (Aβ)₁₂

(Aβ)₁₂ was prepared according to a known method. Specifically, synthetic Aβ₁₋₄₂ peptide (Biopeptide Co.) was suspended at room temperature in 100% 1, 1, 1, 3,3,3-hexafluoro-2-propanol (HFIP). After incubation for 30 minutes, HFIP was removed by evaporating under mild nitrogen flow. Then, Aβ₁₋₄₂ was suspended again in dimethyl sulfoxide (DMSO) at a concentration of 5 mM and then diluted with mM 2-[4-(2-hydroxyethyl)-1-piperazinyl]ethanesulfonic acid (HEPES; pH 7.4) to a final concentration of 110 μM. The resulting solution was incubated at 37° C. for 24 hours while stirring at 500 rpm using a micro stir bar. After centrifugation at 4000 g for 30 minutes, thus obtained 56-kDa Aβ₁₋₄₂ dodecamer, i.e. (Aβ)₁₂, was concentrated (50 kDa and 100 kDa cut-off). The presence of (Aβ)₁₂ in the concentrated sample was identified by sodium dodecyl sulfate polyacrylamide gel electrophoresis (SDS-PAGE). Its spherical structure was confirmed by atomic force microscopy. For cell culturing experiment, a 2 μM solution of (Aβ)₁₂ was diluted using serum-free Dulbecco's modified Eagles medium (DMEM) to a final concentration of 33 nM, which corresponds to a monomer concentration of about 400 nM.

<Adamantane Derivatives>

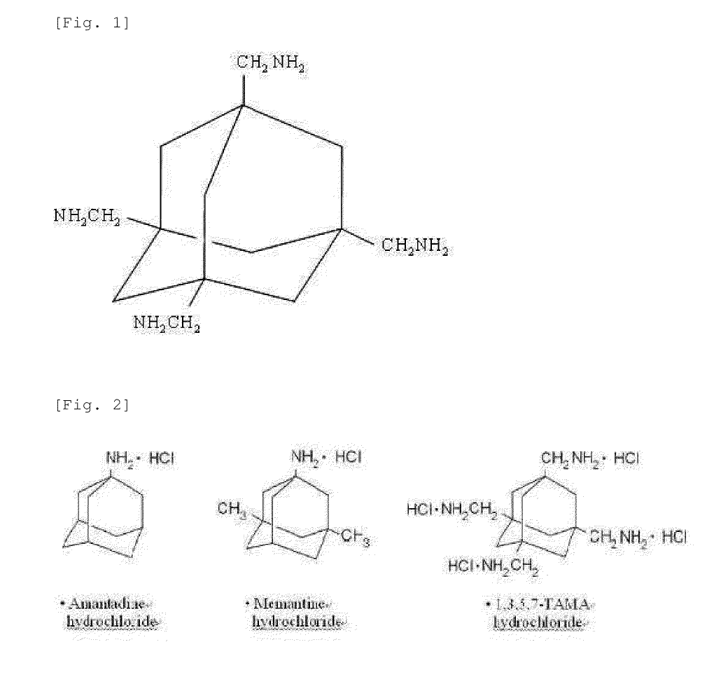

FIG. 1 shows a molecular structure of an adamantane derivative selected as a target in the present disclosure. 1,3,5,7-Tetrakis(aminomethyl)adamantane (hereinafter, TAMA) or TAMA tetra(hydrochloride) was synthesized based on a method disclosed in the literature (Lee et al., Org. Lett. 6, 1705-1707, 2004). The compound structure was identified by TLC, ¹H and ¹³C NMR, MS, or the like.

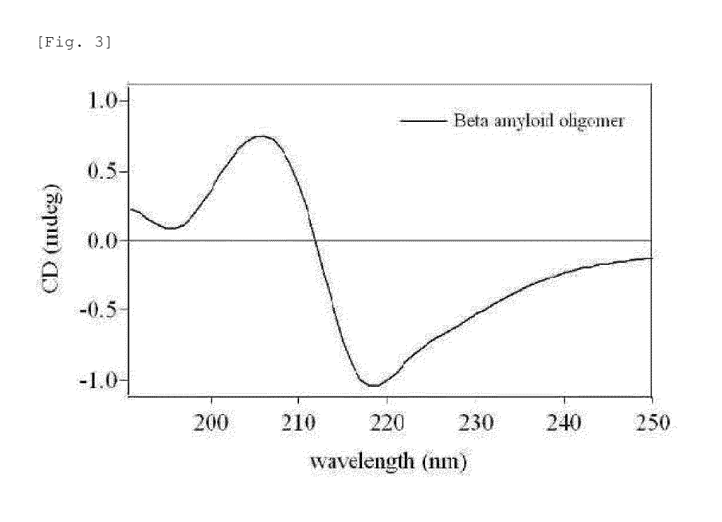

FIG. 2 shows molecular structures of three adamantane derivatives tested in the present disclosure. Amantadine (Sigma) and memantine (Sigma) were selected for comparison with TAMA. Amantadine is known to have antiviral activity, and memantine acts as an antagonist of N-methyl-D-aspartate (NMDA) receptor regulating glutamate activity. In the present disclosure, TAMA was designed to have a high propensity for ionization while having a highly symmetrical 3-dimensional structure, such that it is capable of effectively reducing the neurotoxicity of Aβ₁₋₄₂ resulting from the hydrophobic structure thereof upon contact with the beta amyloid oligomer.

<Cell Culturing and Preparation>

Rat hippocampal H19-7 cells were stored in DMEM containing 10% (v/v) fetal bovine serum (FBS). The cells were cultured to 80% colonies on a culture dish at low concentration in a 35° C. incubator of humidified 5% CO₂ atmosphere. For fluorescence lifetime imaging microscopy (FLIM), the cells were cultured for a day after being transferred to a dish equipped with a poly-D-lysine-coated cover glass (MatTek Corporation). Before FLIM measurement, the cells were stained with 3 μM Cell Tracker Green CMFDA (Molecular Probes) solution and then incubated for 30 minutes in serum-free medium.

<Structural Analysis of Amyloid Oligomers>

Circular dichroism (CD) spectra are useful in determining the secondary structure of proteins in solution. CD spectrum was obtained using a Jasco-810 instrument at high-sensitivity mode (5 mdeg), with a time constant of 4 s, at a scan rate of 100 nm/min, for 30 cycles. The spectrum was obtained at 37° C. in the wavelength region from 190 nm to 250 nm.

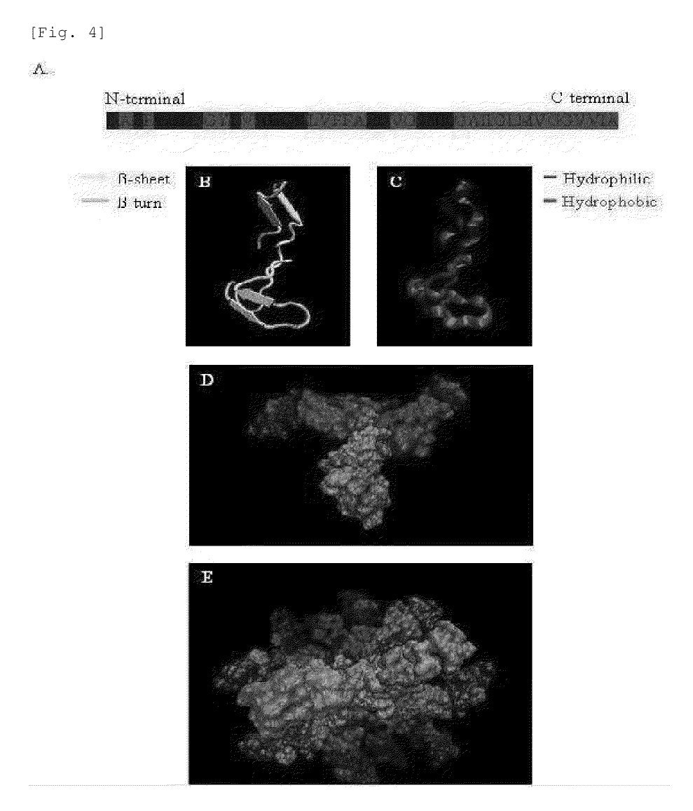

FIG. 3 shows a CD spectrum of (Aβ)₁₂ in HEPES buffer (pH 7.4).

Referring to FIG. 3, absence of negative peaks at 222 nm and 208 nm reveals that the amyloid oligomer is almost free of random coil structure or α-helical structure. Instead, the distinct positive band at about 205 nm and the negative band about 218 nm show that the oligomer includes β-sheet and β-turn structures. A variety of β-turns are known in peptide or proteins. It is not easy to specifically analyze the β-turn structure found from the CD spectrum with other secondary structures, but the experimental data show a very close similarity to the β-turn structure of type I. Thus, it seems that (Aβ)₁₂ consists of an inner core with the hydrophobic C-terminus forming a β-sheet structure and the hydrophilic external surface forming a β-turn structure.

Molecular dynamics (MD) simulation was performed using Accellys Insight II software and an all-atom force field CVFF. The MD simulation was carried out for a fully extended Aβ₁₋₄₂ peptide in vacuum under the condition of constant temperature (300 K) and normal pressure (1 atm) for 100 ns with 1-fs time steps. Based on the structure obtained from the vacuum condition, MD simulation was further carried out to obtain a structure in aqueous solution.

FIG. 4 shows the result of MD simulation. The three-dimensional structure of (Aβ)₁₂ has never been studied in detail as yet. The inventors studied it via MD simulation of the Aβ₁₋₄₂ peptide. schematic representations of an Aβ monomer, an Aβ trimer and an Aβ dodecamer obtained from conformational and MD computation of an Aβ₁₋₄₂ monomer. In the peptide sequence (A), hydrophilic and hydrophobic residues are shown in blue and red colors, respectively.

Referring to FIG. 4, conformational change occurred immediately after the simulation started because the initial extended conformation of the Aβ₁₋₄₂ monomer was very unstable. The structure obtained from MD simulation at 300 K (B) shows how the β-sheets (yellow arrows) and β-turns (azure bands) are configured in the Aβ₁₋₄₂ peptide. For comparison, the hydrophilic and hydrophobic residues were shown in colors (C). As a result of the MD simulation, the basic unit of the amyloid oligomer was identified as a trimer.

The Aβ trimer is shown in D. It can be seen that the hydrophobic C-terminus serves as a motif for forming the oligomer.

The Aβ₁₋₄₂ dodecamer is shown in E. It can be seen that the hydrophobic C-termini cluster to form a spherical core whereas the hydrophilic N-termini are exposed to outside and interact with water molecules. The β-structures and non-covalent bondings stabilize the compact spherical oligomer.

Accordingly, it can be seen that beta amyloid has a bent shape with a stabilized oligomer structure. It is known that amyloid oligomers with different forms share a common structure, which exhibits toxicity for neurons. Based on this fact and the above MD simulation result, it can be inferred that amyloid oligomers generally haven-turns exposed to outside surface, and the β-turns have neurotoxic activity.

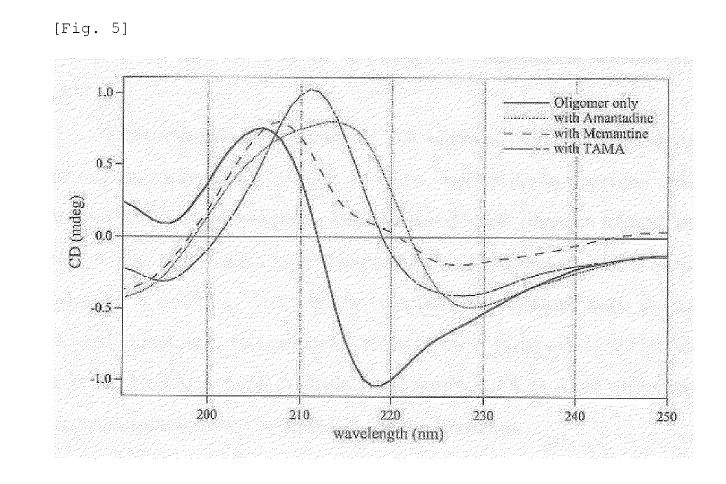

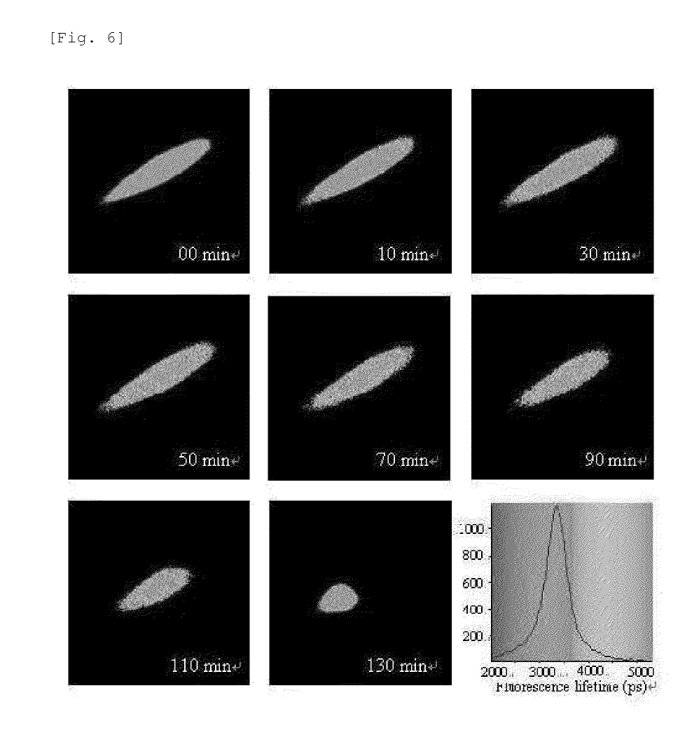

FIG. 5 shows change in the CD spectrum of (Aβ)₁₂ after treatment with adamantane derivatives.

Referring to FIG. 5, when the amyloid oligomer was treated with the adamantane derivatives, the intensity of the positive band of the oligomer at 195 nm decreased and the positive band at 205 nm shifted toward longer wavelength. This means that the adamantane derivatives induce deformation of the oligomer structure by changing the configuration of the β-sheets. Through a separate ThT binding assay experiment, the inventors confirmed that the adamantane derivatives do not turn the amyloid oligomer into the fibrillar form. Rather, the change of the CD spectrum by the adamantane derivatives shows a pattern. The spectrum of the Aβ oligomer treated with memantine is almost identical to that of the typical β-turn peptide. Amantadine results in the broadening of the positive band from 205 nm to 215 nm. Although a detailed structure of the deformed oligomer is not clear, the decrease of intensity at 205 nm is due to the β-turn structure. The oligomer treated with TAMA shows a negative band near 195 nm, which is characteristic of the random coil peptide. To conclude, among the adamantane derivatives tested, TAMA showed the best effect of deforming the β-turn structure, with amantadine and memantine following in order (TAMA>amantadine>memantine).

<FLIM of Single Live Cell>

FLIM is a powerful tool for producing clear images of live cells. FLIM images were obtained at 40 MHz at an excitation wavelength of 467 nm (Picoquant PDL 800-B), using a band pass filter for 500-580 nm and a long pass emission filter for 473 nm (Semlock). A Nikon confocal microscope equipped with an oil-impregnated objective lens (NA 1.3) was used as a platform of an AFM scanner (PSIA). In FLIM measurement, time-correlated single photon counting is very important. B&H S830 was used and 256×256 pixel images were obtained at a scan rate of 1 Hz. The measurement was carried out after placing live cells on a poly-d-lysine-coated cover glass bottomed dish (MatTek Corporation) coated with and then filling serum-free medium diluted with PBS buffer.

Change in fluorescence lifetime was observed in the early stage of apoptosis. Although this result is not insignificant, a detailed description will be omitted since it does not seem to be directly related to the present disclosure. That is to say, FLIM was employed only to obtain clear images of cell morphology.

High-quality images of individual single live cell were obtained through FLIM. For FLIM imaging, mouse hippocampal cells (H19-7) were labeled with Cell Tracker Green having a relatively small pKa value. In all physiological pH's, the single live cell showed bright green fluorescence. The used dye had a fluorescence lifetime of about 3.3 ns.

FIG. 6 shows real-time FLIM images of the single hippocampal cell treated with (Aβ)₁₂. The images were obtained at the corresponding time after treating the cell with 33 nM (Aβ)₁₂.

Referring to FIG. 6, it can be seen that the extended dendrites of the hippocampal neurons treated with the oligomer were reduced. This demonstrates that the neuronal death at the early stage is caused by the toxicity of (Aβ)₁₂. When a similar experiment was performed in the absence of (Aβ)₁₂, the neurons stayed alive at the same time scale without any degeneration.

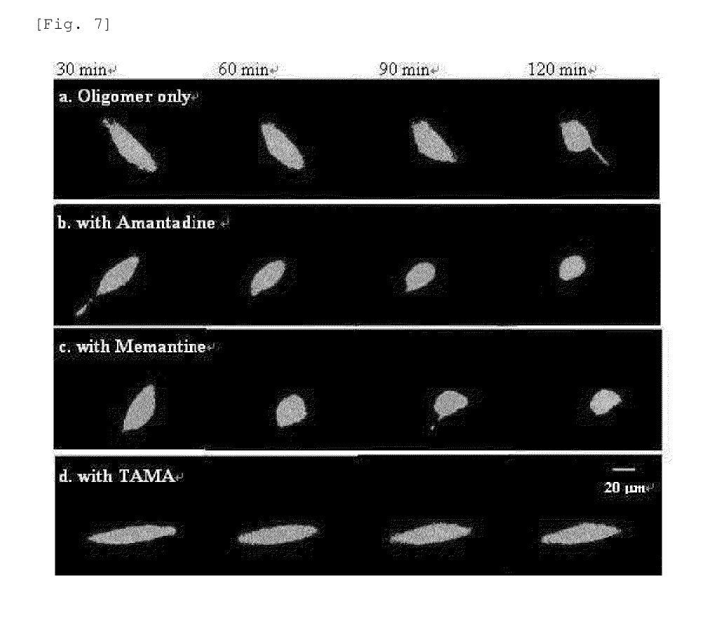

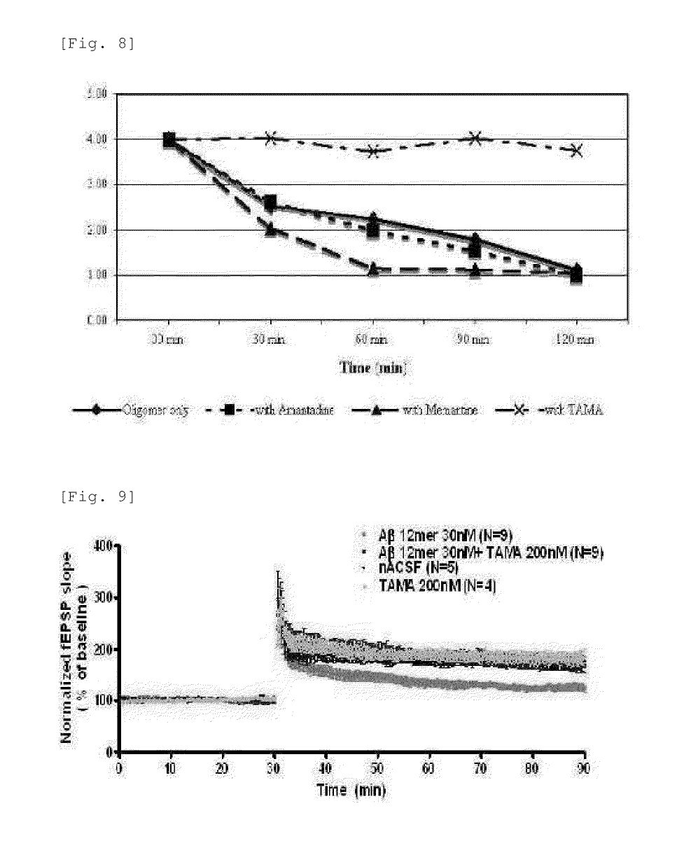

FIGS. 7 and 8 show the FLIM result for the live H19-7 cell in the presence of (Aβ)₁₂ and adamantane derivatives. Specifically, FIG. 7 shows fluorescence images and FIG. 8 shows an aspect ratio of the single H19-7 cell.

Referring to FIG. 7, when amantadine or memantine was added, the apoptosis of the single cell was comparable to or faster than in the presence of (Aβ)₁₂ only. However, when TAMA was present, the single cell treated with the beta amyloid oligomer was not affected by the toxicity of the oligomer at all. In general, the degree of degeneration of neurons can be qualitatively analyzed based on the change in the aspect ratio of a single cell. The progression of neuronal death could be evaluated from the decrease in the aspect ratio.

Referring to FIG. 8, when the neuron was treated only with the beta amyloid oligomer or with the beta amyloid oligomer and amantadine, the aspect ratio decreased to 1.0 after 120 minutes as the cell was reduced. This means that dendrites or synapses do not exist any more. Furthermore, when the cell was treated with the beta amyloid oligomer and memantine, the aspect ratio decreased to 1.0 in 60 minutes.

In contrast, the single cell treated with the oligomer and TAMA showed no decrease in the aspect ratio until 120 minutes. This means that the apoptosis did not proceed. To conclude, TAMA has a remarkably better effect of reducing the toxicity of the beta amyloid oligomer than memantine or amantadine and is capable of preventing apoptosis.

<Electrophysiological Test>

Field excitatory postsynaptic potential (fEPSP) was measured in the CA1 region (Schaffer collateral pathway) of the hippocampus of an ICR mouse using the extracellular recording technique.

Hippocampal slice for fEPSP experiment was obtained from young (4-7 weeks old) male ICR mice. The mice were anesthetized with isoflurane prior to decapitation. After quickly taking out the brain of the mouse from the skull, the brain was cooled with ice and then put in a highly concentrated sucrose solution (sucrose 201 mM, KCl 3 mM, NaH₂PO₄ 1.25 mM, MgCl₂ 3 mM, CaCl₂ 1 mM, NaHCO₃ 26 mM and D-glucose 10 mM) of pH 7.3 with oxygen (95% O₂ and 5% CO₂) supplied thereto. After isolating the hippocampus from the brain, transverse slices were obtained by cutting with a vibratome (Ted Pella, Redding, Calif., USA) in the high-concentration sucrose solution. Before extracellular recording, the transverse slice was incubated at 30° C. in a normal artificial cerebrospinal fluid (nACSF; NaCl 126 mM, KCl 3 mM, NaH₂PO₄ 1.25 mM, MgSO₄ 1.3 mM, MgSO₄ 1.3 mM, CaCl₂ 2.4 mM, NaHCO₃ 26 mM and D-glucose 10 mM) of normal artificial cerebrospinal fluid (nACSF; NaCl 126 mM, KCl 3 mM, NaH₂PO₄ 1.25 mM, MgSO₄ 1.3 mM, MgSO₄ 1.3 mM, CaCl₂ 2.4 mM, NaHCO₃ 26 mM, D-glucose 10 mM) of pH 7.3 with oxygen supplied thereto. After 1 hour, the transverse slice was put in a holding chamber at room temperature for over 1 hour. Then, the transverse slice was transferred to an immersion-type recording chamber while continuously spraying oxygenated nACSF of 28-30° C. at a rate of 2 mL/min.

Extracellular recording was performed by recording fEPSP for two transverse slices at the same time in the same recording chamber from the stratum radiatum of the hippocampal CA1 region. The recording was performed using a glass electrode (tip impedance=1-2 MΩ) immersed in nACSF. Baseline synaptic response was induced by stimulation of the Schaffer collateral pathway using a concentric bipolar stimulation electrode (diameter=75 μm, FHC) at 0.067 Hz (0.15 ms duration). The magnitude of the stimulation was adjusted to a level such that fEPSP with half-maximum slope could be obtained.

In order to induce long-term potentiation (LTP), high-frequency stimulation (HFS, 2 trains, 100 Hz, 1s duration, 15 intervals) was applied with the same strength as that of the baseline experiment. Average LTP was calculated by averaging 20 fEPSP slopes at 55 to 60 minutes after the application of the high-frequency stimulation.

Extracellular field potential was recorded using a microelectrode AC amplifier model 1800 (A-M Systems, Inc., USA). The response signal was low-pass filtered at 5 kHz, digital sampled at 10 kHz using a Digidata 1322 A/D converter, and then analyzed using pCLAMP 9.0 software (Axon Instruments). The experimental data were averaged as means±SEM. The difference between two means was analyzed by an unpaired Student's t-test. p<0.05 was evaluated as significant.

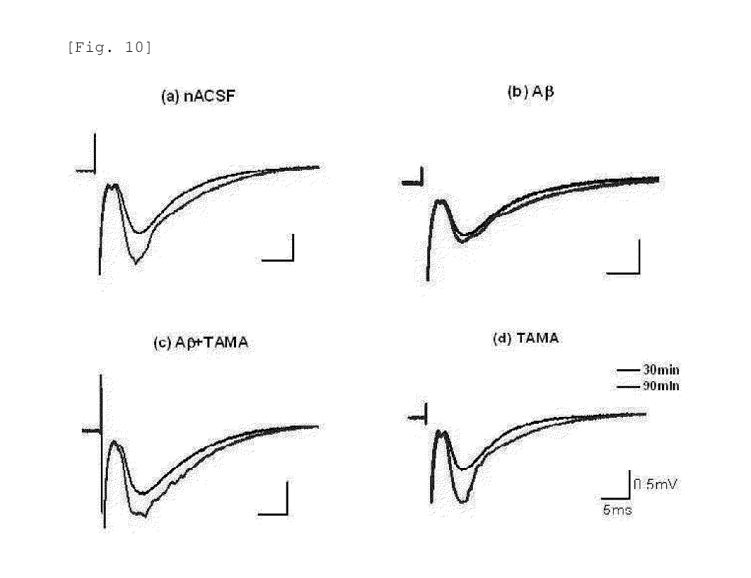

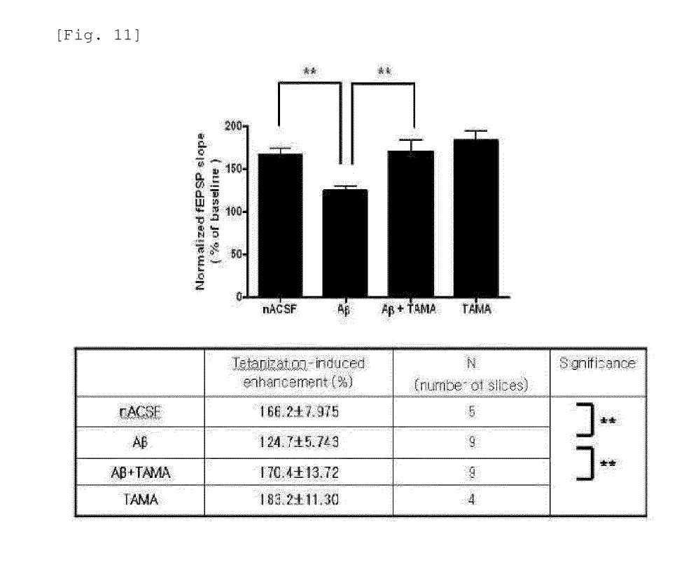

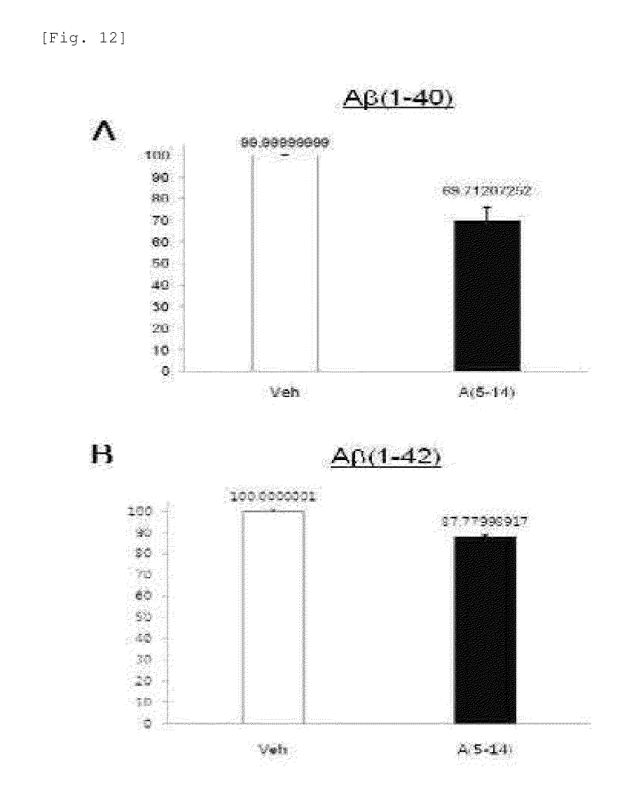

FIGS. 9, 10 and 11 show the result of the fEPSP experiment for the transverse slice of the mouse hippocampal CA1 region. Specifically, FIG. 9 shows a release curve depicting LTP induced by HFS as percentage of the baseline potential, FIG. 10 shows a sweep curve of the mouse hippocampal slice before (30 min) and after (90 min) applying HFS, and FIG. 11 shows an fEPSP slope 1 hour after applying HFS to the hippocampal slice.

After incubating the hippocampal transverse slice in four nACSF solutions for over 1 hour, the respective nACSF solutions were sprayed on the transverse slice to induce LTP. For example, FIG. 9 shows LTP of the transverse slices respectively treated with nACSF solution only (black dots), 30 nM Aβoligomer (dodecamer) dissolved in nACSF solution (red dots), 30 nM Aβ oligomer (dodecamer) and 200 nM TAMA dissolved in nACSF solution (blue dots), and 200 nM TAMA dissolved in nACSF solution (yellow dots).

In control test 1 [see FIG. 9 (black dots), FIG. 10 (a) and FIG. 11 (nACSF)], fEPSP slope was observed 1 hour after applying HFS to the transverse slice treated only with the nACSF solution. It increased by 166.2±7.975% (N=5) with respect to the baseline fEPSP and was maintained over 1 hour. Thus, it can be seen that a significant LTP was induced.

In contrast, the fEPSP slope increased only by 124.7±5.743% (N=9) with respect to the baseline 1 hour after HFS was applied to the transverse slice treated with the 30 nM Aβ oligomer solution [see FIG. 9 (red dots), FIG. 10 (b) and FIG. 11 (Aβ)]. Thus, it can be seen that the LTP of the transverse slice was suppressed by the Aβ oligomer (**p<0.005, unpaired Student's t-test).

Referring to FIG. 9 (blue dots), FIG. 10 (c) and FIG. 11 (Aβ+TAMA), it can be seen that the suppression of the LTP by the Aβ oligomer is prevented by TAMA. Specifically, for the transverse slice incubated for over 1 hour in the 30 nM Aβ oligomer and 200 nM TAMA solution, the fEPSP slope increased by 170.4±13.72% (N=9)1 hour after the application of HFS with respect to the baseline. Thus, it can be seen that addition of TAMA to the Aβ oligomer inhibits the suppression of the LTP by the Aβ oligomer (**p<0.005).

In control test 2 [see FIG. 9 (yellow dots), FIG. 10 (d) and FIG. 11 (TAMA)], wherein the transverse slice was incubated for over 1 hour in 200 nM TAMA solution without containing the Aβ oligomer, the fEPSP slope increased by 183.2±11.30% (N=4) with respect to the baseline 1 hour after the application of HFS. This shows that LTP was induced well as in control test 1 wherein only the nACSF solution was used.

To conclude, whereas LTP of the transverse slice of the hippocampal CA1 region treated with the 30 nM Aβ oligomer solution was significantly reduced as compared to the transverse slice untreated with the oligomer, when the transverse slice of the hippocampal CA1 region was treated with 200 nM TAMA along with the oligomer, the suppression of LTP inducement by the oligomer was effectively prevented.

<In Vivo Test>

Transgenic APPswe/PS1dE9 (Tg-APP/PS1) mouse overexpressing human APP and PS1 mutations was crossbred with hybrid mouse of C57BL6 and C3H until the same genetic background as the original Tg-APP/PS1 mouse was attained. The mice were accommodated in a cage with temperature and humidity controlled and 12 hour night/day cycles (lighting began at 7 a.m.), 1 to 3 mice per cage. The mice were freely given water and feed. All the mice were handled according to the Guideline for Animal Breeding and Management of the College of Pharmacy, Ewha Womans University.

After intraperitoneally injecting a 3.5:1 mixture of ketamine (50 mg/mL) and xylazine hydrochloride (23.3 mg/mL) to the mouse, at 1.0 μg per g body weight, the mouse was anesthetized and placed on a stereotaxic apparatus (Stoelting Company, Wood Dale, Ill., USA) for intracerebroventricular (ICV) injection. 4 μL of a vehicle or 800 nM (318.592 μg/μL) TAMA solution was injected into the right lateral ventricle at a rate of 0.5 μL/min (total TAMA administration amount=1.274 μg). The stereotaxic coordinates of the microinjection were Aβ0.0, ML −1.0 and DV −2.6 in mm units with respect to the bregma. After placing the mouse on a warm pate until it wake up, the mouse was returned to its original cage. 24 hours later, the mouse was sacrificed and brain tissue was taken for enzyme-linked immunosorbent assay (ELISA).

Quantification of Aβ by ELISA was performed according to the literature of Lee et al. (Lee et al., Neurobiol. Dis. 22, 10-24). The anterior cortical tissue was homogenized in Tris-buffered saline (20 nM Tris, 137 mMNaCl, pH 7.6, protease inhibitor). The homogenized suspension was separated into a supernatant containing soluble Aβ and pellets including insoluble Aβ using an ultracentrifuge operating at 100,000 g and 4° C. The supernatant was kept at −70° C. for further analysis. After extracting the insoluble Aβ by adding 70% formic acid (1 mL) to the pellets, the insoluble Aβ was centrifuged again at 100,000 g and 4° C. Then, after neutralizing the insoluble Aβ dissolved in formic acid by adding 1 M Tris-C1 buffer (pH 11.0), ELISA was carried out.

Aβ₁₋₄₀ peptide and Aβ₁₋₄₂ peptide concentrations were determined using Sigma Select™ Human β Amyloid Aβ1-40 and Aβ1-42 colorimetric sandwich ELISA kits (Signal Select™, BioSource, Camarillo, Calif., USA).

FIG. 12 shows the result of the in vivo experiment on Tg-APPswe/PS1dE mice. A and B respectively show the quantity of insoluble Aβ₁₋₄₀ (A) and insoluble Aβ₁₋₄₂ (B) in the prefrontal cortex of a 7.5-month-old Tg-APP/PS1 mouse to which TAMA was administered represented as % mol with respect to when only the vehicle was administered. Experiment was carried out in two groups. The vehicle group consisted of 5 males and 3 females, and the TAMA group consisted of 2 males and 4 females.

Referring to FIG. 12, the quantity of Aβ₁₋₄₀ detected from the brain of the Tg-APPswe/PS1dE9 mouse to which TAMA was administered was only 69.7% that of the vehicle (control) group mouse. Likewise, the quantity of Aβ₁₋₄₂ detected from the brain of the Tg-APPswe/PS1dE9 mouse to which TAMA was administered was only 87.8% that of the vehicle (control) group mouse. Thus, it can be seen that the level of Aβ₁₋₄₀ and Aβ₁₋₄₂ could be significantly reduced even with a single injection of TAMA.

1. A use of 1,3,5,7-tetrakis(aminomethyl)adamantane or a pharmaceutically acceptable salt thereof which induces structural deformation of a beta amyloid oligomer via a strong interaction with the beta amyloid oligomer and thereby reduces toxicity of the beta amyloid oligomer.

2. The use according to claim 1, wherein the 1,3,5,7-tetrakis(aminomethyl)adamantane or the pharmaceutically acceptable salt thereof reduces toxicity of the beta amyloid oligomer in a disease selected from a group consisting of Alzheimer's disease, Parkinson's disease, Huntington's disease, macular degeneration and prion disease.

3. A pharmaceutical composition comprising 1,3,5,7-tetrakis(aminomethyl)adamantane or a pharmaceutically acceptable salt thereof as an agent for reducing toxicity of a beta amyloid oligomer.

4. The pharmaceutical composition according to claim 3, which is for preventing or treating a disease selected from a group consisting of Alzheimer's disease, Parkinson's disease, Huntington's disease, macular degeneration and prion disease.

5. The pharmaceutical composition according to claim 3, which comprises 1,3,5,7-tetrakis(aminomethyl)adamantane tetra(hydrochloride).

6. A use of a dendritic molecule based on 1,3,5,7-tetrakis(aminomethyl)adamantane or a pharmaceutically acceptable salt thereof which induces structural deformation of a beta amyloid oligomer via a strong interaction with the beta amyloid oligomer and thereby reduces toxicity of the beta amyloid oligomer.

7. The use according to claim 6, wherein the dendritic molecule based on 1,3,5,7-tetrakis(aminomethyl)adamantane or the pharmaceutically acceptable salt thereof reduces toxicity of the beta amyloid oligomer in a disease selected from a group consisting of Alzheimer's disease, Parkinson's disease, Huntington's disease, macular degeneration and prion disease.

8. A pharmaceutical composition comprising a dendritic molecule based on 1,3,5,7-tetrakis(aminomethyl)adamantane or a pharmaceutically acceptable salt thereof as an agent for reducing toxicity of a beta amyloid oligomer.

9. The pharmaceutical composition according to claim 8, which is for preventing or treating a disease selected from a group consisting of Alzheimer's disease, Parkinson's disease, Huntington's disease, macular degeneration and prion disease.

10. The pharmaceutical composition according to claim 8, wherein the salt of the dendritic molecule based on 1,3,5,7-tetrakis(aminomethyl)adamantane is a salt with hydrochlorides of the same number as that of amine groups in the molecule added thereto.
